(19) United States
(12) Patent Application Publication
(10) Pub. No.: US 2024/0245872 A1
(43) Pub. Date: Jul. 25, 2024

(54) TEMPERATURE CONTROL METHOD OF VAPORIZER AND SUBSTRATE PROCESSING APPARATUS

(71) Applicant: Tokyo Electron Limited, Tokyo (JP)

(72) Inventors: Junichi HARADA, Nirasaki City (JP); Takuma SHIDO, Oshu City (JP)

(21) Appl. No.: 18/417,507

(22) Filed: Jan. 19, 2024

(30) Foreign Application Priority Data
Jan. 24, 2023 (JP) 2023-008748

(51) Int. Cl. A61M 11/04

(52) U.S. Cl. CPC A61M 11/042; A61M 2205/3372

(57) ABSTRACT

A temperature control method of a vaporizer that is provided with a heater to heat and vaporize an inflowing chemical liquid, includes determining an input power to the heater at a current time, based on an inflow rate of the chemical liquid into the vaporizer at each time in a first prediction interval from a current time to a first predetermined future time, a measured temperature of the vaporizer at the current time, and a predicted temperature of the vaporizer at each time in the first prediction interval.

### CROSS-REFERENCE TO RELATED APPLICATION

This application is based upon and claims the benefit of priority from Japanese Patent Application No. 2023-008748, filed on Jan. 24, 2023, the entire contents of which are incorporated herein by reference.

### TECHNICAL FIELD

The present disclosure relates to a temperature control method of a vaporizer and a substrate processing apparatus.

### BACKGROUND

There are cases where a film forming apparatus as a substrate processing apparatus uses a direct liquid injection (hereinafter abbreviated as “DLI”) vaporizer as a process gas supplier (see e.g., Non-Patent Document 1). In the DLI vaporizer, the vaporizer is maintained at a target temperature (hereinafter referred to as a “set temperature”) by a heater or the like, and heats and vaporizes an inflowing chemical liquid, thereby generating a process gas.

In the related art, the DLI vaporizer keeps input power of the heater of the vaporizer constant, regardless of whether or not the chemical liquid is input, and thus the temperature of the vaporizer is maintained at the set temperature.

### PRIOR ART DOCUMENTS

### Non-Patent Documents

Non-Patent Document 1: “Direct liquid injection”, [online], Horiba, Ltd., [Retrieved on Dec. 28, 2022], Internet <URL: https://www.horiba.com/jpn/semiconductor/key-technologies/element-technology/vaporization-methods/l>

### SUMMARY

According to one embodiment of the present disclosure, there is provided a temperature control method of a vaporizer that is provided with a heater to heat and vaporize an inflowing chemical liquid. The temperature control method includes determining an input power to the heater at a current time, based on an inflow rate of the chemical liquid into the vaporizer at each time in a first prediction interval from a current time to a first predetermined future time, a measured temperature of the vaporizer at the current time, and a predicted temperature of the vaporizer at each time in the first prediction interval.

### BRIEF DESCRIPTION OF DRAWINGS

The accompanying drawings, which are incorporated in and constitute a part of the specification, illustrate embodiments of the present disclosure, and together with the general description given above and the detailed description of the embodiments given below, serve to explain the principles of the present disclosure.

FIG. 1 is a diagram for explaining a DLI vaporizer to which a temperature control method of a vaporizer according to an embodiment of the present disclosure is applied and peripheral devices of the vaporizer.

FIGS. 2A to 2D are diagrams for explaining model predictive control.

FIG. 3 is a graph for explaining a difference between model predictive control and PID control.

FIG. 4 is a block diagram illustrating a concept of model predictive control applied to the temperature control method of the vaporizer according to the present embodiment.

FIG. 5 is a flowchart illustrating the temperature control method of the vaporizer according to the present embodiment.

### DETAILED DESCRIPTION

Reference will now be made in detail to various embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, systems, and components have not been described in detail so as not to unnecessarily obscure aspects of the various embodiments.

In recent years, a film forming process that uses multiple process gases has frequently been used and thus, it is necessary even for a film forming apparatus to be equipped with multiple vaporizers. In the film forming apparatus, there is a demand for miniaturization of vaporizers in order to easily arrange multiple vaporizers.

As a vaporizer becomes smaller, the heat capacity of the vaporizer decreases, so the influence of a chemical liquid due to vaporization heat increases. If input power of a heater is kept constant, the temperature of the vaporizer changes significantly according to change in an inflow rate of the chemical liquid, which is to be vaporized, flowing into the vaporizer (hereinafter referred to as “vaporization flow rate”).

Therefore, controlling the input power of the heater can be considered to maintain the temperature of the vaporizer at a set temperature using PID control, which is a general type of feedback control.

However, since PID control starts adjustment of the input power of the heater after the temperature of the vaporizer starts to change from the set temperature, it takes a certain amount of time for the temperature of the vaporizer, which has changed from the set temperature, to return to the set temperature. In particular, if there is a large change in the vaporization flow rate when the chemical liquid starts to flow or stops flowing, a long time is required for the temperature of the vaporizer to return to the set temperature.

If it takes time for the temperature of the vaporizer to return to the set temperature, for example, in a case where the temperature of the vaporizer has decreased, the amount of the chemical liquid that cannot be vaporized increases, thereby resulting in increased loss of the chemical liquid. Additionally, since the time for which the vaporizer undergoes a large temperature change becomes longer, there is a problem that the vaporizer itself deteriorates more quickly.

In contrast, a technique according to the present disclosure quickly stabilizes the temperature of the vaporizer to the set temperature using model predictive control.

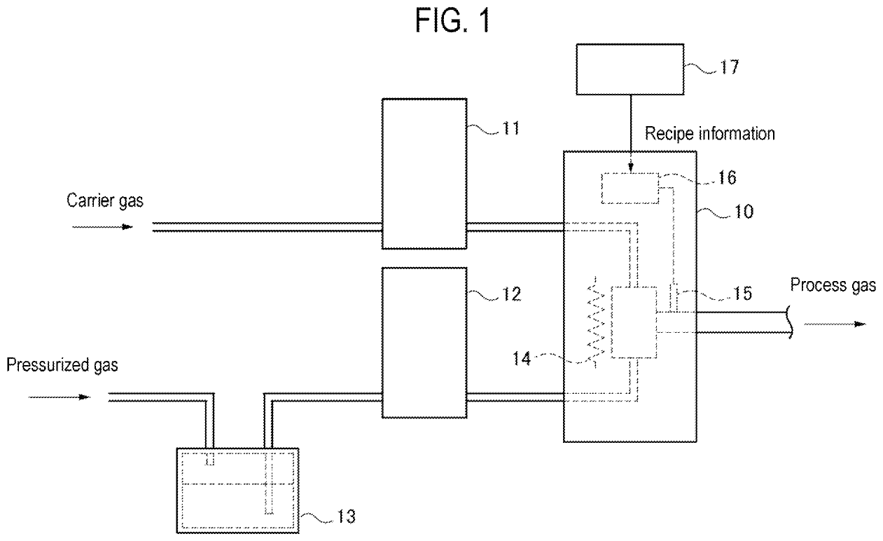

Hereinafter, one embodiment of the technique according to the present disclosure will be described with reference to the drawings. FIG. 1 is a diagram for explaining a DLI vaporizer to which a temperature control method of a vaporizer according to an embodiment of the present disclosure is applied and peripheral devices thereof. In FIG. 1, a mass flow controller (MFC) 11 and a MFC 12 used for liquid are connected to a DLI vaporizer 10. A carrier gas is supplied to the MFC 11 from a tank (not illustrated). The MFC 12 used for liquid is supplied with a chemical liquid extruded from a chemical liquid tank 13 by the pressure of a pressurized gas.

The vaporizer 10 includes a heater 14, a thermometer 15, and a controller 16. Electric power is input to the heater 14 from the outside. The thermometer 15 measures a temperature of the vaporizer 10 (hereinafter simply referred to as “vaporizer temperature”) and transmits the temperature measurement result to the controller 16. The controller 16 determines an input power to the heater 14 (hereinafter simply referred to as “heater input power”) so as to maintain the vaporizer temperature at a set temperature based on the temperature measurement result of the thermometer 15 and recipe information acquired from an external memory 17. Here, the recipe information is, for example, recipe information for film forming processing executed in a film forming apparatus (a substrate processing apparatus) including the vaporizer 10 and contains a vaporization flow rate at each time, which is preset.

The MFC 11 controls an inflow rate of the supplied carrier gas flowing into the vaporizer 10. The MFC 12 used for liquid controls an inflow rate of the supplied chemical liquid flowing into the vaporizer 10. The vaporizer 10 vaporizes the supplied chemical liquid by spraying the chemical liquid into a built-in vaporization chamber (not illustrated) and mixes the vaporized chemical liquid with the carrier gas, thereby generating a process gas. In this case, the vaporizer 10 heats the chemical liquid by the heat of the heater 14 to implement stable continuous vaporization. Further, the vaporizer 10 may not include the controller 16, and an external controller may determine the heater input power based on the temperature measurement result of the thermometer 15 and the recipe information.

When the vaporizer 10 determines the heater input power based on the temperature measurement result of the thermometer 15, PID control, which is general feedback control, is used to start adjustment of the heater input power after the vaporizer temperature has changed from a set temperature. Accordingly, it takes a certain amount of time for the vaporizer temperature, which has changed from the set temperature, to return to the set temperature. Therefore, the present embodiment uses model predictive control, which is a type of feedback control, instead of PID control.

Model predictive control (MPC) is a control method that performs optimization while predicting a future response at each time in a prediction interval. In MPC, a predictive model indicating dynamic characteristics of a control object is set, and a future behavior of the control object in a prediction interval, which is a finite interval from a current time, is predicted. As the predictive model, a step response model, an impulse response model, a transfer function model, an autoregressive moving average model (ARX model), or a discrete state equation is used.

Next, a relationship between an output of the control object (hereinafter referred to as “control output”) and an input to the control object (hereinafter referred to as “control input”) will be described. In MPC, if it is desired to make the control output follow (track) a target value, a time series of control input, which minimizes the area of a tracking error corresponding to the sum of products of differences between a target value and control output at each time in a prediction interval from a current time to a predetermined future time, is searched for.

For example, in MPC, it is assumed that a control input at time t is u[t], a control output at time t is y[t], and a discrete state equation shown in Equation (1) below is used as the predictive model. In Equation (1) below, C and D are weights, which are determined through prior experiments or the like.

$$y [ t + 1 ] = Cy [ t ] + Du [ t ] \qquad ( 1 )$$

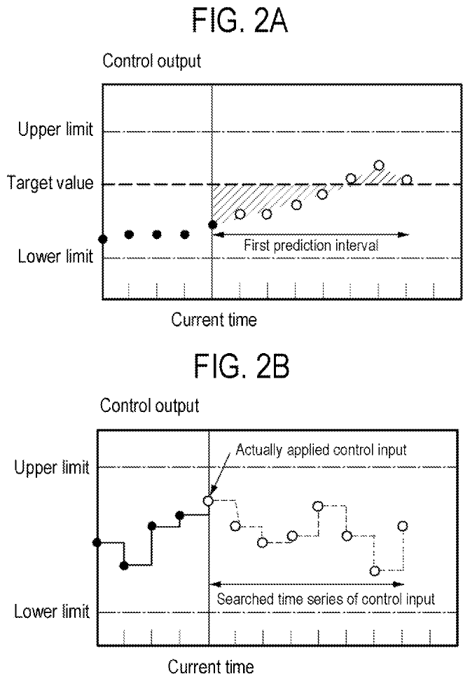

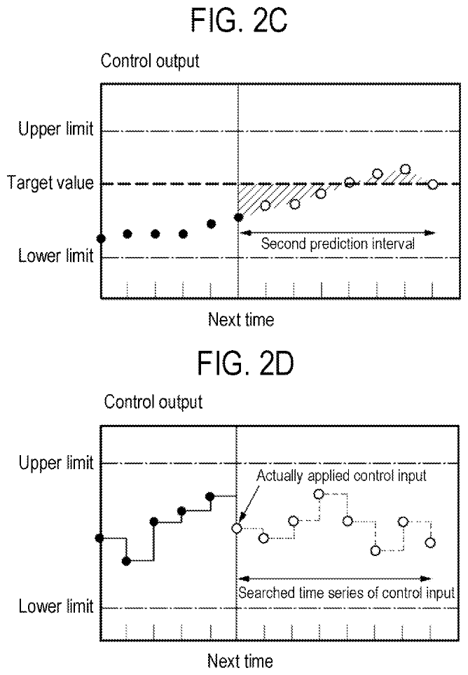

First, a control output at a next time is calculated based on Equation (1) above using a control output at a current time and a temporarily set control input at the current time. The control output at the current time used herein is an actual measured value. Next, a control output at another next time after the next time is calculated based on Equation (1) above using the calculated control output at the next time and a temporarily set control input at the next time. The above process is repeated to calculate a control output at each time in a first prediction interval from the current time to a predetermined future time. In addition, search is performed on control input at each time in the first prediction interval, which minimizes the area of a tracking error between a target value at each time in the first prediction interval and the calculated control output at each time in the first prediction interval. Here, the control input at each time is a value that can be changed. In MPC, the control input at each time (see each white circle in FIG. 2B) is adjusted so as to minimize the area of a tracking error (a hatched area in FIG. 2A). That is, the control input (a time series of the control input) at each time in the first prediction interval, which minimizes the area of the tracking error, is searched for. The search for the time-series of the control input is a kind of optimization problem. Then, a first element of the time series of the control input obtained through the search is applied to a control object as an actual control input at the current time.

Further, at a next time after a predetermined sampling time elapses from a current time, a control output at another next time after the next time is calculated based on Equation (1) above using a control output at the next time and a temporarily set control input at the next time. The control output at the next time used herein is an actual measured value. Next, a control output at another predetermined future time from the another next time after the next time is calculated based on Equation (1) above using the calculated control output at the another next time after the next time and a temporarily set control input at the another next time after the next time. The above process is repeated to calculate a control output at each time in a prediction interval from the next time to a predetermined future time (hereinafter referred to as a “second prediction interval). In addition, search is performed for a control input at each time in the second prediction interval, which minimizes the area of a tracking error (a hatched area in FIG. 2C) between a target value at each time in the second prediction interval and the calculated control output at each time in the second prediction interval. Then, a first element of a time series of the control input (see each white circle in FIG. 2D) obtained through the search is applied to the control object as an actual control input at a next time.

The above process is repeated each time a predetermined sampling time elapses, and a control input at each time is determined using a control output, which is an actual measured value at each time. When determining a control input at a next time after the predetermined sampling time has elapsed from a current time, since an actual measured value of the control output at the next time, which is a result of applying the control input of the current time to the control object, is used, MPC may be said to be feedback control.

A sampling time or the length of a prediction interval in MPC is arbitrary and is set according to dynamic characteristics of the control object. For example, for control objects, fluctuations of which are difficult to converge, the sampling time or the prediction interval is set to be relatively long.

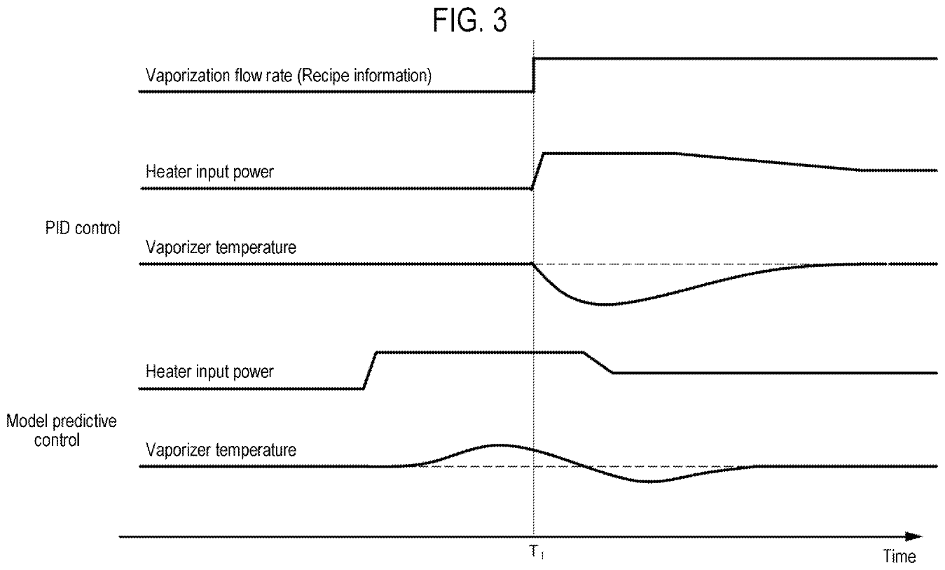

FIG. 3 is a graph for explaining a difference between MPC and PID control. Here, it is considered that a vaporization flow rate of the vaporizer 10 increases at time T1. In this case, at time T1, the heat of the vaporizer 10 (the heater of the vaporizer 10) is taken away by vaporization of a chemical liquid, and thus a vaporizer temperature decreases.

When controlling the vaporizer temperature using PID control, a heater input power starts to be adjusted after the vaporizer temperature starts to change from a set temperature (see broken lines in the figure) at time T1. That is, since adjustment of the heater input power is started late, it takes some time for the vaporizer temperature, which has changed from the set temperature, to return to the set temperature.

On the other hand, when controlling the vaporizer temperature using MPC, if time T1 is included in a prediction interval, the influence of an increase in the vaporization flow rate of the vaporizer 10 at time T1 is reflected in the vaporizer temperature, which is a control output, by a predictive model. That is, the influence of an increase in the vaporization flow rate of the vaporizer 10 at time T1 may be anticipated before time T1, and the heater input power starts to be adjusted before time T1 based on the anticipated result. As a result, the vaporizer temperature, which has changed, may be quickly stabilized to the set temperature. However, since adjustment of the heater input power is started before time T1, the vaporizer temperature starts to change to the set temperature before time T1. In addition, in MPC, since the heater input power is adjusted by anticipating the influence of an increase in the vaporization flow rate of the vaporizer 10, the amount of change in the vaporizer temperature in MPC becomes smaller than the amount of change in the vaporizer temperature in PID control.

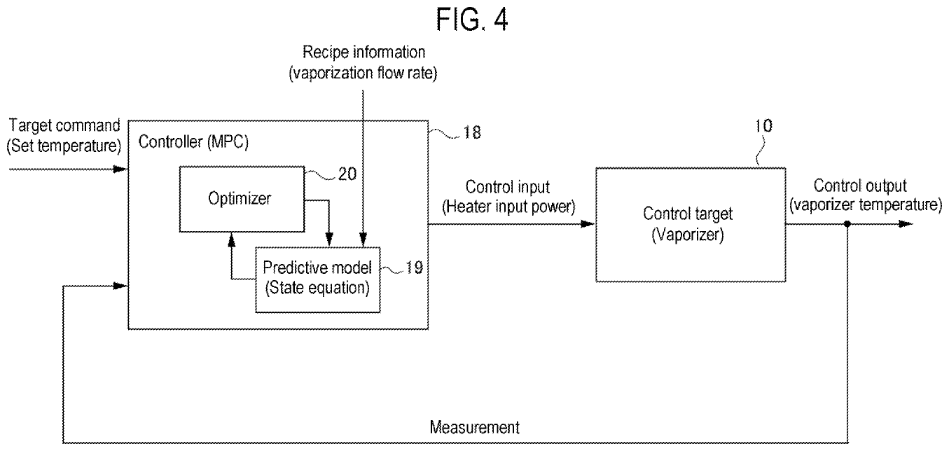

FIG. 4 is a block diagram illustrating a concept of MPC applied to the temperature control method of the vaporizer according to the present embodiment. In FIG. 4, a controller 18 that executes MPC is configured in the controller 16 and includes a predictive model 19 and an optimizer 20. Further, a set temperature of the vaporizer 10 in a prediction interval is input to the controller 18 as a target command.

The predictive model 19 is a model that predicts the temperature of the vaporizer 10 serving as a control object and, as shown in the figure, the predictive model 19 is a model to which a vaporization flow rate at each time in a prediction interval, obtained from recipe information, is input. The predictive model 19 is expressed, for example, as a discrete state equation shown in Equation (2) below. In Equation (2) below, A, B, and V denote weights.

$$x [ t + 1 ] = Ax [ t ] + Bu [ t ] + Vuf [ t ] \qquad ( 2 )$$

In Equation (2) above, u[t] denotes a heater input power, which is a control input, at time t, x[t] denotes a vaporizer temperature, which is a control output at time t, and uf[t] denotes a vaporization flow rate at time t. Here, x[t] is an actual measured value of the vaporizer temperature at time t. Further, since the predictive model 19 has a term corresponding to the vaporization flow rate, in MPC in this embodiment, the vaporizer temperature may be predicted by considering the influence of change in the vaporization flow rate. Furthermore, in Equation (2) above, uf[t] is expressed as a function of time t but, in reality, uf[t] is a predetermined value in the recipe information, so uf[t] is treated as a constant term.

The optimizer 20 is a block for solving an optimization problem. A solution to the optimization problem used by the optimizer 20 is not particularly limited. In this embodiment, the optimizer 20 searches for a time series of the heater input power, which minimizes the area of a temperature tracking error of the heater 14 (predicted from the predictive model 19) for the set temperature of the heater 14 in the prediction interval, by adjusting the heater input power, which is the control input, at each time.

Then, the controller 18 inputs a first element of the time series of the heater input power, obtained through the search, to the vaporizer 10 as the heater input power to be actually input. Further, the vaporizer temperature (control output) as a result of inputting the heater input power is measured and fed back to the controller 18.

That is, in MPC in this embodiment, the vaporization flow rate at each time in the prediction interval is obtained from the recipe information, and the heater input power that should actually be input is determined using the actual measured value of the vaporizer temperature.

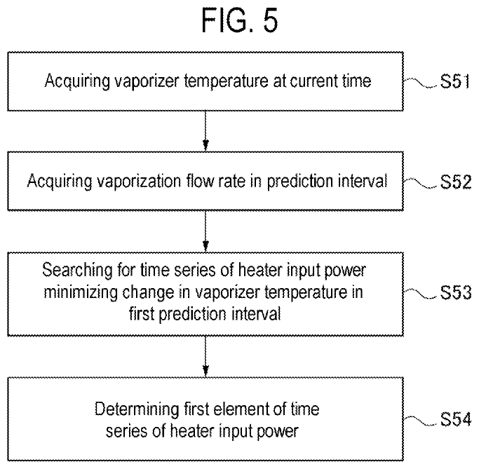

FIG. 5 is a flowchart illustrating a temperature control method of the vaporizer according to the present embodiment. The temperature control method illustrated in FIG. 5 is executed, for example, by the controller 16 of the vaporizer 10 according to a predetermined program. Furthermore, in this embodiment, before executing the temperature control method illustrated in FIG. 5, a predictive model represented as Equation (2) above is previously identified through experiments using the vaporizer 10 or a model similar thereto. In this case, the weights A, B, and V in Equation (2) above are determined.

In FIG. 5, first, a measured vaporizer temperature at a current time is acquired (step S51), and a vaporization flow rate at each time in a first prediction interval is acquired from recipe information (step S52).

Next, a vaporizer temperature at a next time is calculated based on Equation (2) above using the acquired vaporizer temperature at the current time, the acquired vaporization flow rate, and a temporarily set heater input power at the current time. Thereafter, a vaporizer temperature at another next time after the next time is calculated based on Equation (2) above using the calculated vaporizer temperature at the next time and a temporarily set control input at the next time. The above processes are repeated, thereby calculating the vaporizer temperature at each time in the first prediction interval.

Then, a time series of the heater input power, which minimizes change in the vaporizer temperature corresponding to the area of a tracking error between a target temperature and the vaporizer temperature in the first prediction interval, is searched for by solving an optimization problem (step S53).

However, considering that Vuf[t] is treated as a constant term in Equation (2) above, Equation (2) may also be considered to be a function of u, which is the heater input power. Here, it is assumed that x(u), which is the calculated vaporizer temperature at each time in the first prediction interval, is denoted as a vector X after being developed over time, a set temperature xref at each time in the first prediction interval is denoted as a vector Xref, and a heater input power at each time in the first prediction interval is denoted as a vector U. In this case, minimizing the change in the vaporizer temperature corresponds to minimizing a quadratic evaluation function expressed by Equation (3) below. In Equation (3) below, Q and P denote weights.

$$Evaluation function : {( Xref - X ( U ) )}^{T} Q ( Xref - ( U ) ) + {U}^{T} P U \qquad ( 3 )$$

In addition, when upper and lower limits are set as constraints on the heater input power, for example, when a maximum value of the heater input power capable of being supplied is set, these constraints are formulated as inequality constraints. In consideration of the constraints, the time series of the heater input power that satisfies the constraints may be searched for by solving an optimization problem with the constraints based on the evaluation function of Equation (3) above.

Further, when Equation (3) is expressed as a scalar, the evaluation function is expressed as a quadratic equation as shown in Equation (4) below, and minimizing the evaluation function corresponds to minimizing the sum of products of values of Equation (4) below calculated at respective times in the first prediction interval.

$$Evaluation function : {Q ( xref - x ( u ) )}^{2} + P {u}^{2} \qquad ( 4 )$$

Next, a first element of the searched time series of the heater input power, which minimizes the evaluation function, is determined as the heater input power to be input at a current time (step S54).

Then, steps S51 to S54 are repeated each time a predetermined sampling time elapses. For example, after determining the heater input power to be input at a current time using a vaporization flow rate at each time in the first prediction interval, a heater input power to be input at a next time is determined using a vaporization flow rate at each time in a second prediction interval. In this embodiment, while the predetermined sampling time is assumed to be, for example, 1,000 msec (1 second), the sampling time may be changed depending on dynamic characteristics of the temperature of the vaporizer 10.

Furthermore, in repeating steps S51 to S54, when determining the heater input power to be input at the next time after the predetermined sampling time has elapsed from the current time, an actual measured value of the vaporizer temperature at the next time is used. This actual measured value of the vaporizer temperature at the next time is a result of inputting the heater input power to be input, determined at the current time, to the vaporizer 10, so the temperature control method of the vaporizer according to the present embodiment may be called feedback control.

According to the present embodiment, when predicting the vaporizer temperature at each time in a prediction interval using MPC, the influence of change in the vaporization flow rate acquired from recipe information may be considered in predicting the vaporizer temperature. In other words, since the influence of change in the vaporization flow rate can be anticipated in advance before the vaporization flow rate changes, adjustment of the heater input power is started before the vaporization flow rate changes. As a result, the vaporizer temperature which has changed may be quickly stabilized to the set temperature. Further, since the vaporizer temperature that has changed is quickly stabilized to the set temperature, for example, the amount of a chemical liquid that cannot be vaporized due to a drop in the temperature of the vaporizer 10 is reduced, and loss of the chemical liquid is reduced. Furthermore, since the time during which the vaporizer 10 undergoes a large temperature change is shortened, deterioration of the vaporizer 10 itself can be suppressed.

Further, in this embodiment, since the heater input power is adjusted by anticipating the influence of an increase in the vaporization flow rate of the vaporizer 10, the amount of change in the vaporizer temperature is reduced, and an input power to the heater 14 used to adjust the temperature of the vaporizer 10 may be decreased.

Furthermore, in this embodiment, when searching for the time series of the heater input power, which minimizes change in the vaporizer temperature, the upper and lower limits of the heater input power may be considered by solving the optimization problem with constraints. That is, the time series of the heater input power that falls between the upper and lower limits of the heater input power may be searched for.

While the exemplary embodiments of the present disclosure have been described above, the present disclosure is not limited to the embodiments described above, and various modifications and changes may be made within the scope thereof.

For example, although the temperature control method of the vaporizer according to the present embodiment has been executed by the controller 16 of the vaporizer 10 according to a predetermined program, the temperature control method of the vaporizer may be executed by an external controller according to the predetermined program.

In addition, the vaporizer 10 to which the temperature control method of the vaporizer according to the present embodiment is applied may be provided not only in a film forming apparatus but also in other types of substrate processing apparatuses, for example, etching apparatuses, that use a process gas.

Further, although, in this embodiment, the discrete state equation has been used as the predictive model for the temperature of the vaporizer 10, the predictive model is not limited thereto and, for example, an ARX model may be used.

In this case, the predictive model is represented as shown in Equation (5) below.

$$x [ t + 1 ] = Ax [ t ] + Bu [ t ] + Vuf [ t ] + A 1 x [ t - 1 ] + B 1 u [ t - 1 ] + V 1 uf [ t - 1 ] + A 2 x [ t - 2 ] + B 2 u [ t - 2 ] + V 2 uf [ t - 2 ] + \qquad ( 5 )$$

In Equation (5), A, B, V, A1, B1, V1, A2, B2, and V2 denote weights.

When predicting a vaporizer temperature at a next time using the ARX model, not only a vaporizer temperature, a heater input power, or a vaporization flow rate at a current time, but also a vaporizer temperature, a heater input power, or a vaporization flow rate at a time before the current time may be considered. Therefore, the ARX model is suitable when it takes time for a chemical liquid to reach the vaporizer 10 from the chemical liquid tank 13 and when change in a vaporizer temperature is delayed with respect to a change in a vaporization flow rate.

According to the present disclosure in some embodiments, it is possible to quickly stabilize the temperature of the vaporizer to the set temperature.

While certain embodiments have been described, these embodiments have been presented by way of example only, and are not intended to limit the scope of the disclosures. Indeed, the embodiments described herein may be embodied in a variety of other forms. Furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

What is claimed is:

1. A temperature control method of a vaporizer that is provided with a heater to heat and vaporize an inflowing chemical liquid, the temperature control method comprising:
determining an input power to the heater at a current time, based on: an inflow rate of the chemical liquid into the vaporizer at each time in a first prediction interval from a current time to a first predetermined future time; a measured temperature of the vaporizer at the current time; and a predicted temperature of the vaporizer at each time in the first prediction interval.

2. The temperature control method of claim 1, further comprising:
searching for a time series of an input power to the heater at each time in the first prediction interval, which minimizes change in a temperature of the vaporizer in the first prediction interval; and
determining a first input power to the heater in the time series as the input power to the heater at the current time.

3. The temperature control method of claim 1, wherein the inflow rate of the chemical liquid into the vaporizer at each time in the first prediction interval is acquired from recipe information of processing executed by a processing apparatus including the vaporizer.

4. The temperature control method of claim 1, further comprising:
determining an input power to the heater at a next time, which is a time after a predetermined sampling time elapses from the current time, based on: an inflow rate of the chemical liquid into the vaporizer at each time in a second prediction interval from the next time to a second predetermined future time; a measured temperature of the vaporizer at the next time;
and a predicted temperature of the vaporizer at each time in the second prediction interval, wherein the measured temperature of the vaporizer at the next time is a result of inputting, to the heater, the determined input power to the heater at the current time.

1. The temperature control method of claim 1, wherein the input power to the heater is determined using model predictive control, and
wherein a predictive model for predicting the temperature of the vaporizer in the model predictive control is a discrete state equation.

6. The temperature control method of claim 1, wherein the input power to the heater is determined using model predictive control, and
wherein a predictive model for predicting the temperature of the vaporizer in the model predictive control is an ARX model.

7. A substrate processing apparatus including a vaporizer to which a temperature control method is applied,
wherein the vaporizer is provided with a heater to heat and vaporize an inflowing chemical liquid, and
wherein the temperature control method includes:
determining an input power to the heater at a current time, based on: an inflow rate of the chemical liquid into the vaporizer at each time in a first prediction interval from a current time to a first predetermined future time; a measured temperature of the vaporizer at the current time; and a predicted temperature of the vaporizer at each time in the first prediction interval.

8. The substrate processing apparatus of claim 7, wherein the temperature control method further includes:
searching for a time series of an input power to the heater at each time in the first prediction interval, which minimizes change in a temperature of the vaporizer in the first prediction interval; and
determining a first input power to the heater in the time series as the input power to the heater at the current time.

9. The substrate processing apparatus of claim 7, wherein the inflow rate of the chemical liquid into the vaporizer at each time in the first prediction interval is acquired from recipe information of processing executed by a processing apparatus including the vaporizer.

10. The substrate processing apparatus of claim 7, wherein the temperature control method further includes:
determining an input power to the heater at a next time, which is a time after a predetermined sampling time elapses from the current time, based on: an inflow rate of the chemical liquid into the vaporizer at each time in a second prediction interval from the next time to a second predetermined future time; a measured temperature of the vaporizer at the next time; and a predicted temperature of the vaporizer at each time in the second prediction interval,
wherein the measured temperature of the vaporizer at the next time is a result of inputting, to the heater, the determined input power to the heater at the current time.

11. The substrate processing apparatus of claim 7, wherein the input power to the heater is determined using model predictive control, and
wherein a predictive model for predicting the temperature of the vaporizer in the model predictive control is a discrete state equation.

12. The substrate processing apparatus of claim 7, wherein the input power to the heater is determined using model predictive control, and
wherein a predictive model for predicting the temperature of the vaporizer in the model predictive control is an ARX model.
